(19) United States
(12) Patent Application Publication
(10) Pub. No.: US 2006/0293603 A1
(43) Pub. Date: Dec. 28, 2006

(54) Medical system with a multi-dot electrode, where the sub-signals are combined into a synthetic reference

(76) Inventor: Hans Strandberg, Sundbyberg (SE)

Correspondence Address: SCHIFF HARDIN, LLP;PATENT DEPARTMENT, 6600 SEARS TOWER, CHICAGO, IL, 60606-6473, US

(21) Appl. No.: 10/520,753

(22) PCT Filed: Mar. 11, 2003
(86) PCT No.: PCT/SE03/00403

§ 371 Date: Sep. 29, 2005

(30) Foreign Application Priority Data
Jul. 10, 2002 (SE) 0202186-3

(51) Int. Cl. A61B 5/04

(52) U.S. Cl. 600/509

(57) ABSTRACT

Medical system for detecting heart events including an electrode lead provided with a multi-dot electrode unit that comprises at least three dot electrodes, said multi-dot electrode unit is adapted to be used for intra-corporal sensing of heart signals. The heart signals sensed by each of the dot electrodes am applied to a processing means where the signals are combined and a synthetic reference signal is determined. The differences between each dot electrode heart signal and the synthetic reference voltage are determined, and an indication signal is formed based upon said differences, wherein said indication signal is used to detect heart events.

### FIELD OF THE INVENTION

The present invention relates to a system according to the preamble of the independent claim.

### BACKGROUND OF THE INVENTION

Electrode bodies adapted for sensing and/or stimulating electrical activities of a heart and provided with a plurality of electrically insulated electrode surfaces at small distances from one another are previously known through e.g. U.S. Pat. No. 6,064,905 and U.S. Pat. No. 4,848,352.

In U.S. Pat. No. 6,064,905 a multi-element tip electrode mapping catheter is known comprising a tip section including a multi-element tip electrode mounted at the distal end of the tip section comprising a plurality of electrode members electrically isolated from one another and arranged such that, during use of the catheter within the heart, at least two different electrode members are capable of contacting the endocardium tissue at one time.

U.S. Pat. No. 4,848,352 relates to a method for cardiac pacing and sensing using combination of electrodes where a cardiac pacing lead is inserted into a heart chamber. The lead carries a plurality of separate electrodes positioned at the distal tip of the lead and electrically isolated from each other. A separate electrical conductor is provided for each electrode. The electrodes may e.g. be entered in an electrical conductive relation in order to allow sensing to take place from any or all of the electrodes.

Signals detected e.g. inside a heart by a conventional heart electrode where the signal is detected either between two electrodes arranged at a distance from each other and close to the distal end (by using a bipolar electrode) or between a single electrode inside the heart and an indifferent electrode arranged e.g. at the housing of the heart stimulator are often influenced by different kinds of interference. Normal interference signals may emanate from human body generated signals such as muscle interference or from unsuitable electromagnetic fields in the patient's environment.

The interference may be reduced by the use of a bipolar electrode system, where a short electrode distance is one reducing factor. This is a reason for using an electrode surface having several small dot electrodes.

However, experiments performed on animals have shown that the difference between the signals obtained from two dot electrodes at a multi-dot electrode unit is difficult to detect when using detectors conventionally used in heart stimulators. The reason is that the signals are too similar. A conventional heart signal detector works on a pair of electrode surfaces and with traditional heart pacemaker signal processing. The traditional signal processing is amplification, band-pass filter and amplitude detection. The detector level relative the original signal amplitude is then most often between 0.5 mV up to 10 mV.

When sensing a heart signal with a lead comprising several small electrodes close to each other, e.g. in a multi-dot electrode unit, there may be a problem to detect the signal if conventional pacemaker signal processing is used because the differential signal may be only a small part of the total signal amplitude. This part of the amplitude may be in the order 0.1 to 1 mV and can often be less than 0.1 mV. Depending on the vector of the heart activity propagation in relation to the electrode surfaces, the differential signal can even be reduced to nearly zero. Due to the fact that the propagation vector of heart activity varies the differential signal amplitude thereby also varies.

One way to optimise signal detection would be to monitor all combinations of electrode pairs and momentarily select the pair with highest amplitude or at least avoid the pair with zero amplitude. As the propagation vector of heart activity continuously varies, the monitoring and optimisation process must be continuously ongoing which is considered difficult to implement.

The object of the present invention is to solve the above-stated problem regarding detection when using small electrodes arranged at a small distance from one another, e.g. when using a multi-dot electrode unit.

### SUMMARY OF THE INVENTION

The above-mentioned object is achieved by the present invention according to the independent claim.

Preferred embodiments are set forth in the dependent claims.

Thus, the object is achieved by creating a synthetic reference signal from all or at least three of the dot electrodes to which the sensed signal from each of the electrodes may be related.

According to the present invention the amplitudes of all (or most of) the dot electrodes are combined to a synthetic reference signal. The only requirement for the signal processing is that there is some difference between any of the dot electrode pairs. As all dot electrodes are grouped together, external interference is totally avoided and high amplification for the differential signal may be used.

The processing means that processes the detected signals may be arranged either close to the multi-dot electrode unit (e.g. in the electrode lead tip) or e.g. in a heart stimulator to which the electrode lead is connected.

Thereby the signal between the synthetic reference signal and any electrode will have a significant amplitude, as the amplitude of the heart signal not can be totally cancelled. The requirement of the electrodes used to create the synthetic reference signal is that not two pairs of electrodes have the same vectors.

This results in a signal separated from zero between any of the electrodes and the synthetic reference signal, and that each signal in relation to the synthetic reference signal will have enough amplitude to contribute for detection and the cancellation phenomenon in bipolar systems, i.e. two dots, is thereby avoided.

The signal processing performed by the processing means is simple and straightforward. There is no need to filter the signals and the unsuitable side effects of the pacemaker filters (may be referred to as “memory-effects” of the filter) used today are avoided.

The present invention accomplishes total avoidance of some of the signal problems in pacemakers as signal detection of different types of interference such as external interference or far field heart signals.

In a preferred embodiment of the present invention an electrode lead is provided with the multi-dot electrode unit and a processing means arranged close to the multi-dot electrode unit and supplies detection signal(s) from the processing means through the lead into the heart stimulator.

### SHORT DESCRIPTION OF THE APPENDED DRAWINGS

FIG. 1 shows a block diagram of the medical system according to a preferred embodiment of the present invention.

FIG. 2 shows a block diagram that schematically illustrates the multi-dot electrode unit and the processing means according to the preferred embodiment.

FIG. 3 shows a diagram of a preferred embodiment as a schematic of electronics of the present invention.

FIG. 4 is a cut through view of a multi-dot electrode unit in the distal tip of an electrode lead.

FIG. 5 is a cross-sectional view of the multi-dot electrode unit in the distal tip of an electrode lead as shown in FIG. 4.

FIG. 6 shows signal recordings from seven dot electrodes of a multi-dot electrode unit placed in the ventricle of an animal.

FIG. 7 shows a small part of the signal recordings as in FIG. 6 in a larger scale.

FIG. 8 shows differences of signal recordings of seven dot electrodes (S1-S7) of a multi-dot electrode unit where electrode S1 is used as a reference.

FIG. 9 shows differences of signal recordings of seven dot electrodes (S1-S7) of a multi-dot electrode unit where the synthetic reference signal (SR) calculated according to the present invention is used as a reference.

FIG. 10 shows three signal tracings of different processed signals.

FIG. 11 shows the same signals as in FIG. 10 but with another time scale.

### DETAILED DESCRIPTION OF PREFERRED EMBODIMENTS OF THE INVENTION

A multi-dot electrode unit used in connection with the present invention is preferably arranged at the distal end of an electrode lead. The multi-dot electrode unit includes a plurality of dot electrodes electrically insulated from one another and arranged at small distances from one another. An exemplary distance between dot electrodes is less than 1 mm.

In its broadest aspect a dot electrode should be regarded as a small electrode surface having an optional geometric shape, e.g. circular, elliptical, rectangular, square, band-shaped etc. In case of a circular dot electrode the diameter is in the interval of 0.05-1 mm.

The distance between adjacent dot electrode surfaces is preferably less than 1 mm. The distance between the most distant dot electrodes in a multi-dot electrode unit is less than 10 mm.

The electrode lead provided with the multi-dot electrode unit will be able to pick up nearly the same electrical signal on each dot electrode.

In order to perform bipolar sensing using the dot electrodes the differential signal (that is the signal between two dot electrodes) is small and a special effort must be taken in the design to avoid so-called common mode effects.

Initially a general overview of the present invention will be given.

According to the present invention the signals from N dot electrodes are added together in order to create an un-normalized synthetic reference signal. N could be any number between 3 and the total number of dot electrodes. The total number of dot electrodes may be very large but a suitable number is between 10 and 30, preferably 20. In one embodiment N is in the range 4-8. The created signal is then divided by N and a normalized synthetic reference signal is obtained. The differential signal to be used for detection is made in relation to the normalized synthetic reference signal.

The following example further illustrates how the synthetic reference signal is created and how a differential signal for a specific dot electrode in relation to the created synthetic reference signal is determined.

The signal amplitude at a dot electrode “i” is designated Uᵢ in relation to a reference. This reference may be obtained from a point capable of providing a long-term stable reference level, e.g. a separate indifferent electrode at the pacemaker housing or the battery in the pacemaker. The un-normalized synthetic reference signal will then be:
Σ(U₁+ . . . +U_{N})

A normalized synthetic reference signal (SR-signal) will then be:
SR-signal=(1/N)×Σ(U₁+ . . . +U_{N})

The differential signal A_{diff(i)} for dot electrode i will then be:
A_{diff(i)}=Uᵢ−(1/N)×Σ(U₁+ . . . +U_{N})

This differential signal is amplified to a suitable detecting level. As the short distance between the dot electrodes essentially reduces the influence of interference the only filtering necessary to perform is to transfer the signal shape to a shape that is easy to detect.

To detect the heart signal from activity near the dot electrodes the contribution from all electrodes is most optimal. As the difference signal of each dot electrode relative the synthetic reference can be both positive and negative the difference signals must be processed prior the signals are combined.

According to a preferred embodiment of the present invention the absolute values of the difference signals are determined and then added together to form an indication signal. This sign conversion may be achieved by rectification. Alternatively and according to an alternative embodiment, the difference signals instead are squared and then added together to form the indict signal. Still a further possibility is to form a value representing the signal contents for the difference signals upon which the indication signal is formed.

According to a preferred embodiment of the present invention the signal processing means is arranged close to the multi-dot electrode unit. As indicated above the multi-dot electrode unit preferably is placed at the distal tip of the electrode lead. However, any position along the electrode lead may be possible without departing from the scope of the present invention as it is defined in the appended claims.

Thus, according to this preferred embodiment the processing means includes a specific amplifier and detector circuit placed close to the electrode tip. In this case a fully integrated circuitry with enough small size to be placed within one or a few cubic millimetres is arranged in the electrode tip.

With regard to the power supply of the processing means two conductors are arranged in the electrode lead from the heart stimulator (that includes a battery) to the electrode tip. One of the conductors may be the one used for heart stimulation. The information from the signal detection may also be sent from the electrode tip to the heart stimulator on the same pair of conductors. By using switched loading an altered voltage level may transfer information in binary format from the electrode tip into the heart pacemaker. The signal transfer does not contain complete information with regard to full signal morphology but only detection information is transferred. The detection information may then be used by control means in the heart stimulator to control e.g. the pulse generation.

The signal processing performed by the processing means to detect heart activity may be done in several ways, for example band pass filtering of the signals, adding signals, correlate signals together etc.

The type of electronics can be of several types, e.g. traditional analogue, analogue to digital converted and then digitally processed, analogue sampled and processed in analogue way by a so-called switch capacitor technique. Thus, the present invention may be implemented in many different ways where some will be described in the following.

The preferred implementation of the processing means in view of technical realisation as micro-power electronics with smallest dimensions to be assembled into the electrode tip is the so-called sampled switch cap technique.

However, for easier understanding of this implementation the description is as if it were traditional analogue technique keeping in mind that there is methods for direct translation between those techniques.

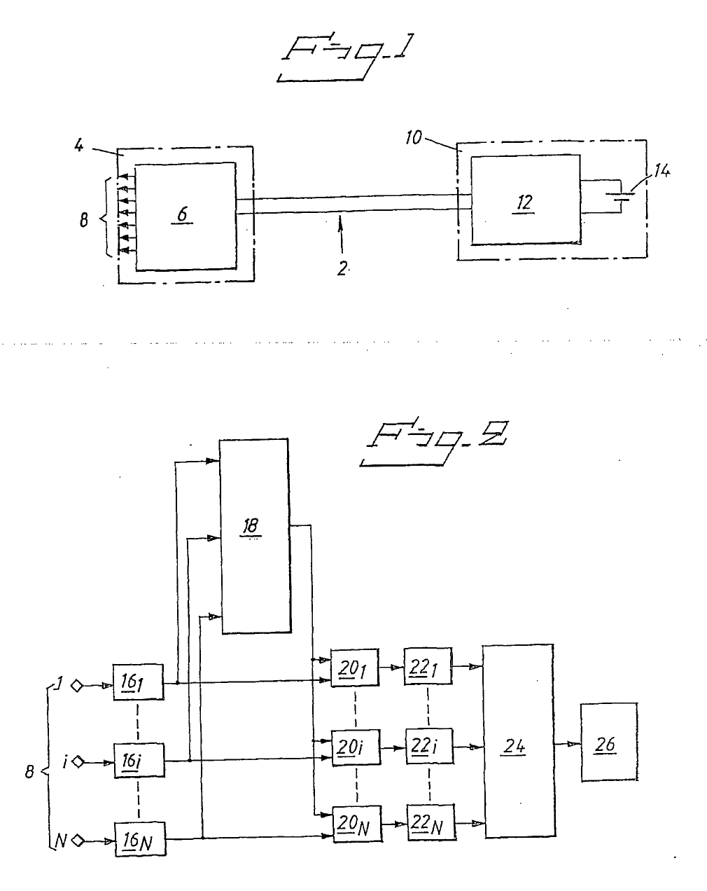

FIG. 1 shows a block diagram of the medical system according to a preferred embodiment of the present invention. The system comprises an electrode lead 2 provided with a multi-dot electrode unit 4 including a processing means 6. The multi-dot electrode unit 4 is provided with a plurality of dot electrodes 8 and may be arranged near or in contact with a heart wall, e.g. in the right ventricle of the heart.

In the figure the multi-dot electrode unit is arranged close to the distal end of the electrode lead, i.e. in the electrode lead tip, but may also be arranged at another position along the lead, e.g. within the form of a cylindrical ring electrode at a predetermined distance from the distal end.

The electrode lead 2 is coupled to an implantable heart stimulator 10 that inter alia comprises heart therapy and control means 12 and power supply means 14.

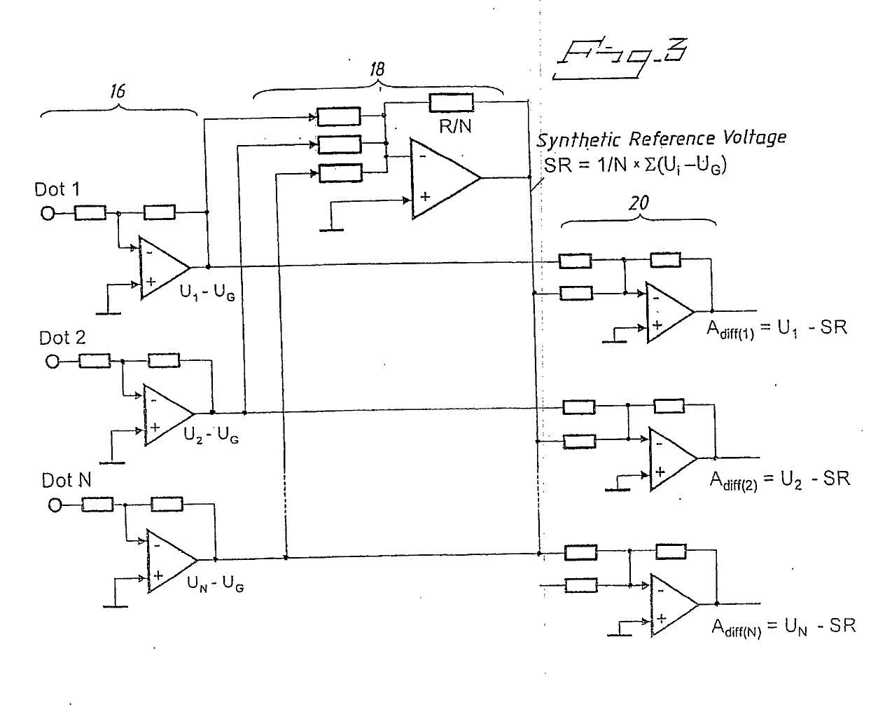

FIG. 2 shows a block diagram that schematically illustrates the multi-dot electrode unit and the processing means according to the preferred embodiment. Each of the dot electrodes 8₁-8_{N} is connected to a respective amplifying means 16₁-16_{N} in order to amplify the dot electrode signals sensed by the dot electrodes.

The signal (U₁-U_{N}) provided to each amplifier 16 must be in relation to any reference voltage. The reference voltage could be any stable voltage potential such as the power supply voltage or a derivate of such voltage.

If the amplitude of a sensed dot electrode signal is in the order of 0.1-2 mV the amplification should be in the range of 50-2000 in order to obtain a dynamic detection voltage in the order of 1 Volt. The amplifying means are connected to a synthetic reference calculation means 18 where a synthetic reference signal (SR-signal) is calculated.

The synthetic reference signal and the amplified dot electrode signals are applied to difference signal means 20₁-20_{N} where differences between each of the amplified dot electrode signals and the synthetic reference signal are formed according to the formula A_{diff(i)}=Uᵢ−SR-signal, where i=1 . . . N.

According to a preferred embodiment of the present invention the difference signals are applied to rectifying means 22₁-22_{N} where the signals are rectified and these rectified signals are in turn applied to a summing means 24. The summing means determines an indication signal that is applied to a detector means 26 provided with predetermined heart event detection criteria that may include one or many threshold(s) representing different predetermined heart events to be detected. One or many detection signals is(are) generated by the detector means as a result of the these comparisons.

The multi-dot electrode unit is in particular suitable for detecting “fast” heart events in the close vicinity of the electrode. If the multi-dot electrode unit is placed in the atrium these fast heart events could be spontaneous or stimulated P-waves and atrial fibrillation or flutter and if the electrode is placed in the ventricle spontaneous or stimulated R-waves could be detected.

According to an alternative embodiment of the present invention the processing means is arranged at a distance from the multi-dot electrode unit, e.g. inside the body in an implantable heart stimulator or outside the body in an external medical device. In this alternative embodiment each dot electrode is connected via a separate conductor in the electrode lead to the processing means located e.g. inside a heart stimulator housing.

FIG. 3 shows a diagram as a schematic of electronics illustrating the circuitry of the preferred embodiment of the present invention. In the illustrated embodiment three of the N dot electrodes are shown. The rectangles represent resistors having the resistance R except where indicated a resistor value of R/N and the triangles represent operational amplifiers.

U₁-U_{N} are the dot electrode potentials in relation to an electrical reference point.

In the figure U_{G} represents a ground potential against which each of the dot electrode potentials are related to. It should be noted that U_{G} is a conducting surface (e.g. a metal surface) close to the multi-dot electrodes (e.g. inside said processing means). This potential (U_{G}) is not stable compared to e.g. a dot electrode since it is related to the battery source and the housing of the implant. However, if this defined ground potential (U_{G}) or zero potential is used internally in the processing means as the earth potential then no additional disturbance or interference is induced during the further processing of the signals.

Using the same reference signs as in FIG. 2 the circuitry in FIG. 3 includes the amplifying means 16 that amplify the sensed heart signals and generate dot electrode signals (U₁-U_{N}), the synthetic reference calculation means 18 that calculates the synthetic reference signal (SR-signal) and the difference signal means 20 that determines the difference signals A_{diff(1)}-A_{diff(N)}.

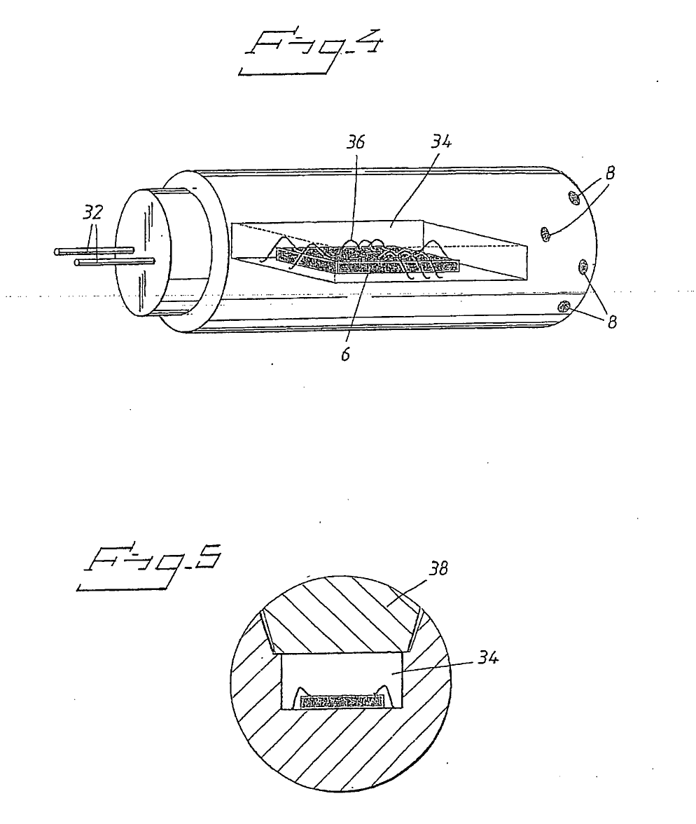

FIG. 4 is a cut through view of a multi-dot electrode unit in the distal tip of an electrode lead with processing means 6 and two conductors 32 connecting the processing means to the control means in the pacemaker. The distal tip is a hermetic capsule of ceramic material with a cavity 34 housing the processing means. The diameter of the capsule is in the order of 3 mm. The two conductors pass through the wall of the capsule where it is connected to the processing means via wire bonding 36. The dot electrodes 8 are evenly spread on the end of the electrode tip and are connected to the processing means.

FIG. 5 is a cross-sectional view of the multi-dot electrode unit in the distal tip of an electrode lead as shown in FIG. 4. The cavity 34 is closed with a lid 38 inserted from above and hermetically sealed to the capsule. The sealing may consist of a low temperature melting glass or some type of metallic soldering.

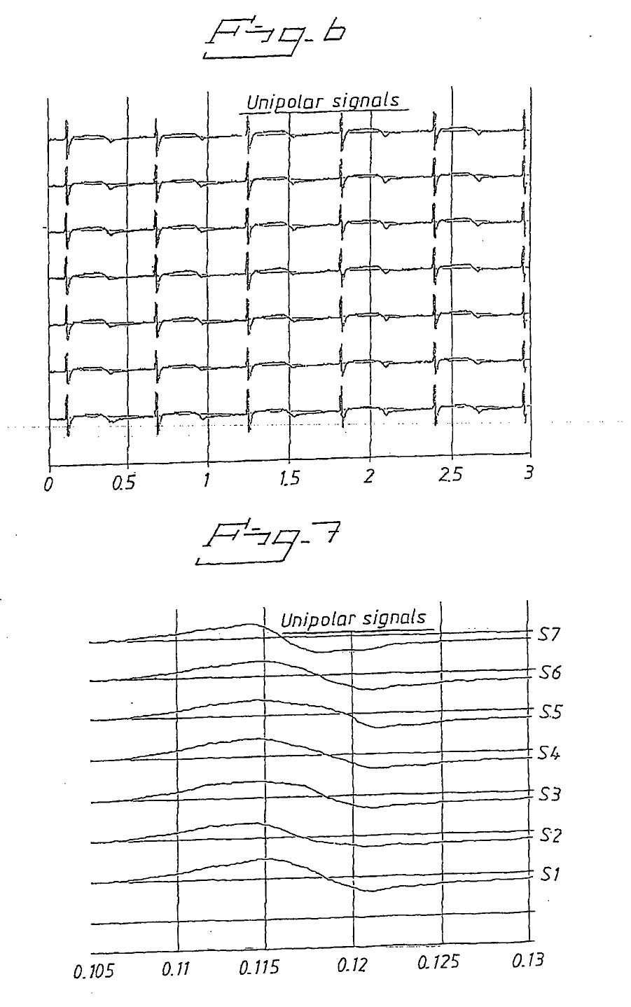

FIG. 6 shows signal recordings from seven dot electrodes of a multi-dot electrode unit placed in the ventricle of an animal where the R-waves easily may be identified. The signals are unipolar which means that they are measured between each of the electrodes and an electrode surface at the capsule of the pacemaker. The time scale (horizontal axis) is from 0 to 3 seconds. The signal from electrode 7 is at the top and electrode 1 at the bottom. Only small non-obvious differences between the electrode signals may be identified.

FIG. 7 shows a small part of the signal recordings as in FIG. 6 in a larger scale (0.105-0.130 seconds). Also here only small differences may be identified.

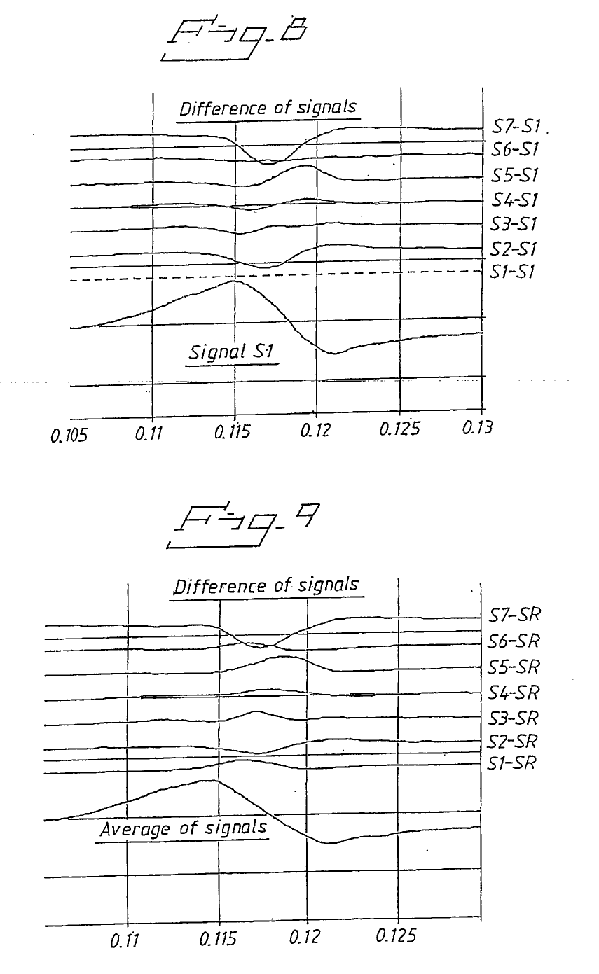

FIG. 8 shows differences of signal recordings of seven dot electrodes (S1-S7) of a multi-dot electrode unit where electrode S1 is used as a reference. The time scale is 0.105-0.130 seconds. The signal S6-S1 is cancelled out because the signals S6 and S1 are too similar. Investigations have shown as a rule that one or two of the bipolar signals are cancelled out. Which pair that is cancelled out cannot be predicted. Even if one pair is good and another is bad the situation may be opposite duets varying conditions such as varying propagation vector or activity. This illustrates that the use of one bipolar signal from the multi-dot electrode unit is not sufficient and if more bipolar signals than one are used selection problems may occur.

FIG. 9 shows differences of signal recordings of seven dot electrodes (S1-S7) of a multi-dot electrode unit where the synthetic reference signal (SR) calculated according to the present invention is used as a reference. The time scale is 0.105-0.130 seconds. The signals S1 to S7 are unipolar R-wave signals from the multi-dot electrode unit placed in the right ventricle of an animal using the indifferent electrode at the housing as reference. The signal at the bottom is the average of the signals, i.e. the synthetic reference signal.

These signal differences (S1-SR, . . . , S7-SR) illustrate that there is a low but significant signal amplitude in all signal recordings.

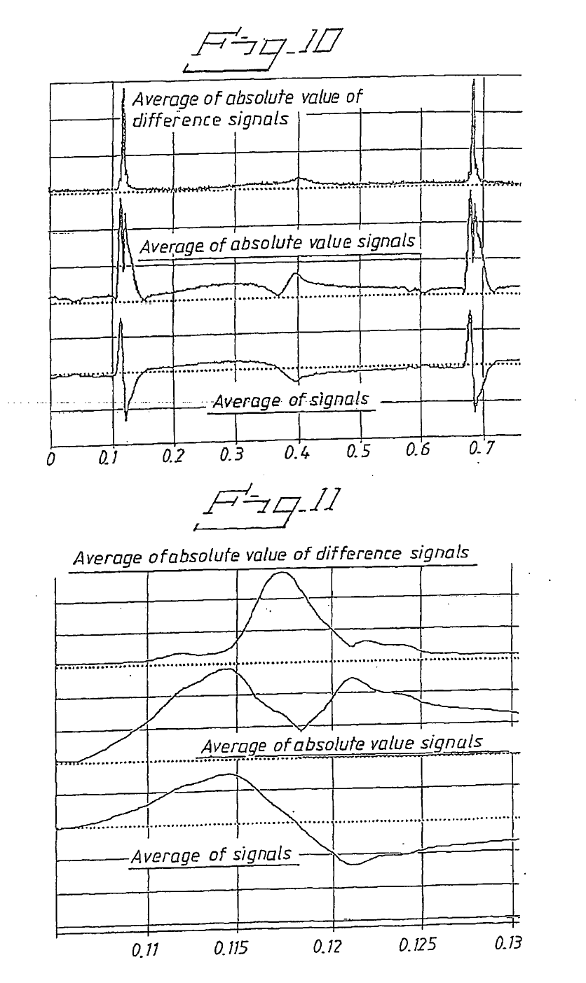

FIG. 10 shows three signal tracings of different processed signals.

The signal at the top show the average of absolute value of difference signals processed according to the whole processing chain as earlier described according to the invention, i.e. all the signal differences created with use of the synthetic reference (S1-SR, . . . , S7-SR) are rectified and then added together. The heart activity is easy to detect, as the pulse amplitude is very distinguished. Detection occurs when the pulse amplitude is higher than a preset detection amplitude threshold (not shown).

The second signal (in the middle) shown in FIG. 10 is included to show that other signal processing also may be used to get a detectable signal result from all the electrode signals, in this case the average of the absolute amplitude values of all unipolar signals. However, the signal processing used according to the present invention achieves a more distinguished result, which has superior detection qualities.

The signal at the bottom of FIG. 10 is the average of the dot electrode potentials, i.e. the synthetic reference signal (SR).

FIG. 11 shows the same signals as in FIG. 10 but with another time scale (0.105 to 0.130 seconds). As in FIG. 10 the signal at the top in the figure illustrates the use of a signal processing according to the present invention. Investigations have shown that this signal shape has less variation than the variation in signal shape of each dot electrode signal, and also less variation than the variations of the average signal.

The present invention is not limited to the above-described preferred embodiments. Various alternatives, modifications and equivalents may be used. Therefore, the above embodiments should not be taken as limiting the scope of the invention, which is defined by the appending claims.

1. Medical system for detecting heart events including an electrode lead provided with a multi-dot electrode unit (4) that comprises at least three dot electrodes (8), said multi-dot electrode unit is adapted to be used for intra-corporal sensing of heart signals,
characterized in that heart signals sensed by each of the dot electrodes are applied to a processing means (18, 20, 22, 24) where the signals are combined and a synthetic reference (SR) signal is determined, the differences between each dot electrode heart signal and the synthetic reference voltage are determined, and an indication signal is formed based upon said differences, wherein said indication signal is used to detect heart events.

2. Medical system according to claim 1, characterized in that the synthetic reference signal (SR-signal) is determined according to the formula:
SR-signal=1/N×Σ(U₁+ . . . +U_{N}), where
N is the number of dot electrodes, and
U₁ . . . U_{N} are the dot electrode potentials in relation to an electrical reference point.

3. Medical system according to claim 2, characterized in that for each dot electrode a differential dot electrode value A_{diff(i)} is determined by the formula:
A_{diff(i)}=Uᵢ−SR-signal, where i=1 . . . N.

4. Medical system according to claim 3, characterized in that said indicating signal is formed by adding together the absolute values of A_{diff(i)}, where i=1 . . . N.

5. Medical system according to claim 3, characterized in that said indicating signal is formed by adding together the squared values of A_{diff(i)}, where i=1 . . . N.

6. Medical system according to claim 3, characterized in that said indicating signal is based upon the signal contents of A_{diff(i)}, where i=1 . . . N.

7. Medical system according to claim 1, characterized in that said indication signal is applied to a discrimination means adapted to generate an detection signal if the indication signal fulfils predetermined heart event detection criteria.

8. Medical system according to claim 1, characterized in that said processing means is arranged in said electrode lead in connection with the multi-dot electrode unit.

9. Medical system according to claim 1, characterized in that said multi-dot electrode unit is arranged at the distal end of said electrode lead.

10. Medical system according to claim 1, characterized in that said synthetic reference signal is determined as the average value of at least three of the detected dot electrode potentials.

11. Medical system according to claim 1, characterized in that said processing means is arranged in an implantable medical device and electrically connected to the multi-dot electrode unit via conducting means in said electrode lead.

12. Medical system according to claim 1, characterized in that multi-dot electrode unit may also be used to apply stimulation pulses to tissue.

13. Medical system according to claim 1, characterized in that said processing means is adapted to change mode of operation for the multi-dot electrode unit between a detection-mode and a stimulation-mode.
